(19) United States
(12) Patent Application Publication
(10) Pub. No.: US 2011/0002851 A1
(43) Pub. Date: Jan. 6, 2011

(54) Cationic Colloidal Carriers for Delivery of Active Agents to the Blood-Brain Barrier in the Course of Neuroinflammatory Diseases

(75) Inventors: Heinrich Haas, Munchen (DE); Paolo Riccio, Bari (IT)

Correspondence Address: MORGAN LEWIS & BOCKIUS LLP, 1111 PENNSYLVANIA AVENUE NW, WASHINGTON, DC, 20004, US

(73) Assignee: MediGene AG, Planegg (DE)

(21) Appl. No.: 12/446,533

(22) PCT Filed: Oct. 31, 2007
(86) PCT No.: PCT/EP07/09460

§ 371 Date: Apr. 21, 2009

Related U.S. Application Data
(60) Provisional application No. 60/856,271, filed on Nov. 3, 2006.

(51) Int. Cl. A61K 49/00; A61K 31/337; A61K 39/395; A61K 38/21; A61K 31/52; A61K 31/675; A61K 31/136; A61K 31/519; A61K 38/07; A61K 31/573; A61K 9/14; A61K 9/127; A61P 25/00; A61P 37/02

(52) U.S. Cl. 424/9.1; 514/449; 424/130.1; 424/85.6; 514/263.2; 514/90; 514/656; 514/262.1; 514/21.9; 514/181; 424/489; 424/450

(57) ABSTRACT

The present invention relates to the use of cationic colloidal compositions for the targeted delivery of an active compound to an inflammatory site or an activated vascular site for the preparation of a medicament for the treatment of MS and in general for all CNS or PNS inflammatory neurodegenerative and demyelinating diseases and for diagnostic applications of such compositions.

The present invention relates to the use of cationic colloidal carrier compositions for the targeted delivery of active compounds to affected sites at the blood-brain barrier (BBB) or the blood-nerve barrier (BNB) for the treatment or diagnosis of neuroinflammatory or neurodegenerative diseases, particularly for the treatment or diagnosis of diseases involving demyelination of neuronal cells.

### BACKGROUND

The blood-brain barrier (BBB) is represented by the complex cerebral vascular endothelium at the interface between the Central Nervous System (CNS) and systemic blood circulation.

The BBB, which presents a restricted permeability to most hydrophilic solutes, is crucial for the maintenance of the homeostasis of the CNS environment and CNS protection for optimal functional activity. The blood-nerve barrier (BNB) is the analogue of BBB in the Peripheral Nervous System (PNS),

In neuroinflammatory diseases such as multiple sclerosis (MS), in the CNS, and Guillain-Barré Syndrome (GBS), in the PNS, these barriers can change dramatically during the early stages of the diseases showing an enhanced permeability and acting as active mediators of the neuroinflammatory processes.

The hallmark of both MS and GBS is the breakdown of the myelin sheath. Myelin is the multilamellar, lipid-rich membrane wrapped around nerve axons to provide segmental insulation. CNS myelin is produced by oligodendrocytes, whereas PNS myelin is produced by Schwann cells.

In the course of MS, which is the most common human disabling neurological disease in young people, the myelin sheath is broken down and many scars disseminated in time and space are produced in the brain as well as in the spinal cord due to an autoimmune inflammatory attack against myelin. The causes of MS remain to be ascertained. What is clear is that MS is a complex multifocal and multifactorial disease: genetic, infectious, immunological and environmental factors have all been taken into consideration as possible causative agents, but none of these factors alone can explain the genesis of this disease. In accord with its complexity, MS shows a marked clinical heterogeneity and is classified in different clinical subtypes: relapsing-remitting MS, the most diffuse, and progressive MS. The latter shows different clinical courses such as primary progressive, secondary progressive and progressive-relapsing.

A MS counterpart in the PNS is the chronic inflammatory demyelinating polyradiculoneuropathy (CIDP). In addition to MS, there are acute, monophasic disorders, such as the above mentioned inflammatory demyelinating polyradiculoneuropathy termed Guillain-Barré syndrome (GBS) in the PNS, and the acute disseminated encephalomyelitis (ADEM) in the CNS. Axonal damage can add to a primarily demyelinating lesion and cause permanent neurological deficits or precede demyelination (Gold et al., 2000).

Useful animal models exist which mimic certain features of human demyelinating diseases: Experimental Autoimmune Encephalomyelitis (EAE) and Experimental Autoimmune Neuritis (EAN) for MS and GBS, respectively.

The understanding of the human disease mechanisms are based in part on the experimental models mentioned above. Other evidence has been obtained by the response to therapy as well as by magnetic resonance imaging (MRI) and other diagnostic procedures (McDonald et al., 2001). MRI and pathological studies have shown that MS lesions are distributed around venules and that the inflammatory damage to blood vessels and disruption of the blood brain barrier (BBB) is an early event, if not the first, in the pathogenesis of MS and in the formation of new focal lesions (Werring et al., 2000).

The inflammation in CNS white matter might be initiated by yδT cell infiltration. Later CD4 and CD8 activated T cells are involved and loss of myelin and axons occurs (Hafler, 2004). In EAE, disease starts when an immunodominant peptide of the injected myelin antigen [myelin basic protein (MBP), proteolipid protein (PLP) or myelin oligodendrocyte glycoprotein (MOG)] is presented by a MHC class II molecule of an antigen presenting cell (APC) to CD4 T cells and activates them.

In MS, disease might start because in a genetically susceptible host, microbes contain protein sequences activating the APCs which can cross-react with self myelin antigens. The effect of this so-called molecular mimicry, together with a defect of immunoregulatory activity related to a decrease of regulatory T cells, leads to the increase of autoreactive T cells.

Myelin-reactive T cells pass the BBB and enter into the CNS, where they enter in contact with microglia, the endogenous APC's in the brain.

Antibody autoreactivity and presence of autoantibodies in MS plaques also has been observed. There is a characteristic increase in oligoclonal IgG in cerebrospinal fluid (CSF).

Due to the inflammatory nature of the described diseases, inflammatory mediators obviously play a major role in the mechanism of said diseases.

Inflammatory mediators include tumour necrosis factor, cytokines, prostaglandins, oxygen radicals and matrix metalloproteinases (MMPs). These latter are very important not only because they are involved in several inflammatory diseases, in particular in the CNS, but also because they can be inhibited and might have an additional regenerative role (Yong, 2005).

In MS, MMPs have the role to facilitate transmigration of circulating leukocytes into the CNS. T cells use MMP-9 to attack the extracellular matrix and capillary basal lamina and cross the BBB. Migration of T cells is inhibited by Interferon-beta by its effect on MMP-9 (Stuve et al., 1997).

Monocytes are also prominent contributors of the neuroinflammation in MS through a mechanism that involves high MMP expression. MMP-9 CSF levels increase in MS and correlate with BBB injury, while improved BBB permeability and decreased MMP-9 in the CSF both occur with steroid treatment.

While an increased expression of MMP-9 in MS has been observed, the concentrations of natural tissue inhibitors (TIMPs) of metalloproteinases (MMPs) are low in MS. MMP-9 is able to degrade the myelin sheath and it has been shown that in MS there is a specific intrathecal synthesis of MMP-9 (Liuzzi et al., 2002). Thus, inhibition of MMPs at the level of BBB leads to an inhibition of leukocyte entry into CNS and the inhibition of myelin breakdown caused by MMPs. It has also been shown that mice that are deficient in MMP-9 are resistant to EAE, the MS animal model.

Further, it has been shown that expression of MMP-9 is dose-dependently inhibited by treatment with the antiviral agents AZT or IDV in LPS-stimulated astrocytes and microglia. These results raise the possibility that AZT and IDV interfere directly with MMP production in glial cells and independently from their antiviral activity, thus suggesting the possible therapeutical use in neurological diseases associated with MMP involvement such as MS (Liuzzi et al., 2004).

Symptoms of MS include: weakness and/or numbness in one or more limbs; tingling of the extremities and tightband-like sensations around the trunk or limbs; dragging or poor control of one or both legs to spastic or ataxic paraparesis; hyperactive tendon reflexes; disappearance of abdominal reflexes; Lhermitte's sign; retrobulbar or optic neuritis; unsteadiness in walking; increased muscle fatiguability; brain stem Symptoms (diplopia, vertigo, vomiting); hemiplegia; trigeminal neuralgia; other pain syndromes; nystagmus and ataxia; cerebellar-type ataxia; Charcot's triad; diplopia; bilateral internuclear opthalmoplegia; myokymia or paralysis of facial muscles; deafness; tinnitus; unformed auditory hallucinations (because of involvement cochlear connections); vertigo and vomiting (vestibular connections); transient facial anesthesia or of trigeminal neuralgia; bladder dysfunction euphoria; depression; fatigue; dementia, dull, aching pain in the low back; sharp, burning, poorly localized pains in a limb or both legs and girdle pains; abrupt attacks of neurological deficit; dysarthria and ataxia; paroxysmal pain and dysesthesia in a limb; flashing lights; paroxysmal itching; and/or tonic seizures, taking the form of flexion (dystonic) spasm of the hand, wrist, and elbow with extension of the lower limb.

A number of approaches were taken to deal with the above mentioned problems (Kieseier and Hartung, 2003).

Treatment of acute relapses is mainly based on glucocorticosteroids and, less frequently, on plasma exchange. Treatment of relapsing-remitting MS is presently based on the use of either:
1) interferon beta (IFNβ), including three different formulations,
2) glatiramer acetate (GA, Copaxone®), a random peptide made up of four amino acids,
3) intravenous immunoglobulins with effect on the immune system;
4) mitoxantrone, an inhibitor of DNA repair and synthesis;
5) azathioprine, an immunosuppressive drug;
6) natalizumab, a recombinant monoclonal antibody against α4 integrins. Trials with natalizumab, however, have been recently suspended.

Nonetheless, natalizumab has been approved or re-approved after intensive analysis of the trials.

Treatment of secondary progressive MS is more limited and based mainly on the use of:
1) IFN β, in the presence of relapses (progressive-relapsing MS);
2) mitoxantrone,
3) cyclophosphamide, a cytotoxic alkylating agent with immunosuppressive effects;
4) methotrexate, an inhibitor of DNA and RNA synthesis;
5) cyclosporin, an anti-inflammatory peptide.

Furthermore, with regard to new developmental approaches focused on remyelinization, it has been recently demonstrated that intravenously injected syngenic adult neural progenitor cells (aNPC) promote multifocal remyelination and functional recovery in mice affected by a chronic-progressive form of EAE.

In addition, since some of the factors that prevent remyelination include physical and molecular barriers such as the astrocytic glial scars, a combination of paclitaxel with vitamin B12 cyanocobalamin has been suggested to enhance remyelination. Astrocytosis was reduced in treated mice. The mechanism of action of the combination therapy was due to activation of endogenous IFN β (Mastronardi and Moscarello, 2005).

In the literature, a number of approaches using carriers or solubilizing agents have been proposed for treating MS. The aims of these approaches were to improve solubility and/or other pharmacokinetic parameters or to protect the compound from undesired interactions with biomolecules. These therapy approaches are mainly focused on the immune system, e.g. the T cells.

One approach for example describes the application of micellar paclitaxel in EAE (Cao et al., 2000). In this therapeutic approach paclitaxel was used as an inhibitor of lymphocyte activation. Paclitaxel in its role as a microtubule stabilizer acts on the cascade of human T cell activation. The publication uses a water soluble formulation of paclitaxel, which is obtained by using a micellar vehicle made of biocompatible block copolymers of poly (DL-lactide-)-block-methoxy-polyethylene glycol. Cao et al. could show that paclitaxel caused a dose-dependent suppression of T cell proliferation.

Faulds et al. also combined a well known drug with a vesicular formulation for the treatment of MS. In DE19739693 they describe the use of IFN β in combination with other active agents in a liposomal formulation. The combination of a drug currently used in the treatment of MS with a liposomal formulation was also pursued by Schmidt et al. (Schmidt et al., 2003). In this therapeutic approach, the glucocorticosteroid Prednisolone was encapsulated in liposomes comprising DPPC, PEG-DSPE and cholesterol and applied in a EAE model. Although the liposome concentration in spleen was magnitudes higher compared to brain and spinal cord, a higher liposome concentration in brain and spinal cord of EAE rats compared to healthy rats was observed.

In WO 98/40049 Bäuerlein et al. describe specific magnetosomes and magneto-liposomes for the diagnosis of MS. In particular, the therapeutic application of magnetosomes is suggested if a therapeutic substance is applied at the same time. However, Bäuerlein et al. do not specifically suggest a therapeutic application of magneto-liposomes for MS but rather for tumor diseases and the liposomes always have to include magnetic particles.

In DE4132345 Eibl et al. describe lytic agents like lysolecithins which are encapsulated in liposomes formed with optionally one negative or positive lipid for the treatment of MS. The liposomal encapsulation hereby is necessary to prevent hemolytic and tissue necrotic side reactions when applying lytic agents intravenously. Eibl et al do not suggest to use a targeting mechanism via liposomal formulation.

In one publication the association between MS lesions and neovascularization is hypothesized (Kirk et al., 2004). It is suggested that several key components in the pathophysiology of MS are also associated with angiogenesis. However, if angiogenesis is involved, it is only a part of the pathological changes due to the inflammation reaction within MS. Kirk et al. suggest the systemic use of anti-angiogenic substances such as minocycline hydrochloride, which belongs to the group of antibiotics or CM101, a Group B Streptoxin that selectively disrupts proliferating endothelium by interaction with the (CM201) receptor. Kirk et al. do not suggest any novel therapeutic concepts for MS except the use of anti-angiogenic drugs.

Since the disclosure of McDonald et al., U.S. Pat. No. 5,837,283 it is known, that positively charged liposomes specifically target angiogenic endothelial cells and chronically inflamed trachea, but not endothelial cells in the brain. McDonald et al. propose the use of cationic liposomes for treating cancer and diseases where angiogenesis plays a key role but not for BBB targeting or treating MS.

Several approaches for BBB targeting are described in the literature including liposomal delivery (See for example (Schnyder and Huwyler, 2005)). However, in that cases, targeted delivery by antibody-functionalized liposomes (immunoliposomes) to molecular ligand moieties, which are characteristic for the BBB, is applied.

Despite strong research efforts, the cause of MS remains elusive, the pathological mechanisms are not fully understood and the clinical course is highly variable. The treatment options are still very limited. A particular disadvantage of today's MS treatment options is the systemic and untargeted application of the drugs. Thus, high concentrations at the inflammatory site can only be obtained by high systemic dosing. This approach is limited by the high costs, the adverse effects of the therapeutic compounds and/or formation of antibodies (Bertolotto, 2004). No therapy approach based on an increased local concentration of the therapeutic compound at the inflammatory site in the CNS or PNS has been described so far.

Thus, the problem underlying the present invention was to provide a new and improved approach for diagnostic and therapeutic applications in inflammatory neurodegenerative diseases like MS.

### DESCRIPTION OF THE INVENTION

In the context of the present application, the use of pharmaceutical compositions comprising colloidal cationic carriers for the targeted delivery of active agents (compounds) to sites of the BBB and BNB with altered molecular and physicochemical properties, particularly inflammatory sites, which become evident within MS and other inflammatory and degenerative diseases of the CNS and PNS, is disclosed. These colloidal cationic carrier compositions may be employed in the diagnosis or treatment of an inflammatory neurological or neurodegenerative disease, e.g. a demyelinating disease. The disease may be associated with, accompanied by or caused by the occurrence of altered or inflammatory sites in the BBB and/or BNB. Further, the disease may be associated with, accompanied by or caused by an autoimmune attack upon the CNS and/or PNS. In contrast to established treatment protocols, administration of colloidal cationic carriers leads to a local action at affected sites of the BBB and BNB.

According to the present invention, the specific localization of an active agent at altered or inflammatory sites results in a selective action of the active agent at these sites. For example, therapeutic agents can be administered, which are capable of limiting the entry of activated T cells into the CNS by blocking the activity of metalloproteinases (MMPs) such as MMP-9 and/or of the activity of oxygen radicals. Other therapeutic agents that can be delivered via this new targeting approach include drugs that are currently used for the treatment of MS like IFNβ, corticosteroids or cytostatic agents. Further, the invention encompasses the targeted administration of diagnostic agents.

Targeted delivery by cationic carriers to altered sites of the BBB and/or BNB can be achieved already at early disease stages, e.g. at an early stage of EAE, even before clinical disease symptoms occur. Particularly at such an early disease stage, it could not have been expected that an angiogenic or inflammatory pathological situation is present such as described in the literature as necessary for cationic targeting. Thus, the invention surprisingly allows diagnosis and treatment of neuroinflammatory disorders at very early disease stages.

Targeting inflammatory sites is even possible where no proliferation at high rate at inflammatory sites occurs. This targeting to inflammatory, but not proliferating endothelial tissue was unexpected and it opens additional new diagnostic and therapeutic opportunities for selective treatment of neurological inflammatory or degenerative diseases associated with the BBB and/or BNB. Delivery of active agents to affected sites of the BBB and/or BNB and optionally through the BBB and/or BNB opens new options for treating neurological diseases such as MS.

Thus, the use of cationic colloidal carriers to specifically target drugs to activated vascular sites of the BBB for the treatment or diagnosis of MS and related pathological situations represents a completely novel concept which has not been disclosed before. The binding of cationic colloidal carriers to the altered luminal plasma membrane of brain endothelial cells enables new therapeutic approaches for the delivery of active agents to or through the BBB and/or BNB in general, e.g. the delivery of anti-inflammatory agents or compounds able to reduce the entry of T cells, macrophages and antibodies.

The benefit of the invention is not restricted to a therapeutic use. The described targeting effect can also be used in diagnostic applications, e.g. by targeting imaging agents to the inflammatory sites at the BBB and/or BNB. Thus, new imaging approaches can be used to improve diagnosis of diseases like MS.

The local action of the active compound at the BBB and/or BNB has a number of advantages with respect to conventional treatment based on the following considerations:
1) The blood brain barrier is the site where the autoimmune attack upon the CNS begins.
   As demonstrated by pathological and MRI studies, disruption and increased permeability of the BBB is the critical early event involved in the inflammatory diseases of the CNS. BBB is indeed the place where the autoimmune attack upon the CNS first begins (Werring et al., 2000).
2) The administration of an active agent in a cationic colloidal carrier composition leads to a local enrichment at the affected sites of the BBB at the same dosing level and thus to an increased overall efficacy due to the higher concentration of the active agent at the site of action.
3) A lower total dose of the active agent might be applied to the patient. The targeting mechanism facilitates a concentration at the site of action which is comparable to the conventional treatment. Reduced dosing will attenuate adverse drug effects.
4) At the same dosing level of a diagnostic agent, the sensitivity of a diagnostic method will be improved.
5) Using a suitable diagnostic agent, altered sites at the BBB can be determined at a very early stage and with high spatial resolution. This enables a more accurate and adequate decision about therapy.

### DEFINITIONS

“About” as used in the present specification describes a deviation from the given value of plus or minus 5%.

“Active agent” or “active compound” refers to an agent or compound that is diagnostically or therapeutically effective or to a combination of diagnostic or therapeutic agents.

“Altered molecular and physicochemical properties” refers to properties that are changed in a pathologic stated compared to a healthy state. Such properties may include but are not limited to the increased expression of cell adhesion molecules, as for example ICAM-1 (CD54) or VCAM-1 (CD106) (Mynagh, P. N. The interleukin-1 signalling pathway in astrocytes: a key contributor to inflammation in the brain (2005), J. Anat 2007, 265-269) by vascular endothelial cells at the pathologic site. Also the permeability of the endothelial cell layer for monocytes, lymphocytes or leukocytes can be increased. In the altered state, the sites of the blood brain barrier have a higher affinity for cationic liposomes as described in Example 2 of the present application. This altered property might be caused by the increased presence of negatively charged fenestrae (Thurston, G. et al. (1998), Cationic liposomes target angiogenic endothelial cells in tumors and chronic inflammation in mice. J Clin Invest 101, 1401-13), an increase of the negative charge density due to overexpression of anionic phospholipids (in particular phosphatidylserine) at the luminal surface (Ran, S., Downes, A. & Thorpe, P. E. (2002), Increased exposure of anionic phospholipids on the surface of tumor blood vessels. Cancer Research 62, 6132-6140), or activation of protein phosphorylation by cytokines such as tumor necrosis factor-alpha (Nwariaku, F. E. et al. (2002), The role of p38 map kinase in tumor necrosis factor-induced redistribution of vascular endothelial cadherin and increased endothelial permeability. Shock 18, 82-5).

“Altered sites of the BBB and/or BNB vasculature” refers to sites of the vascular endothelium that constitutes the BBB or BNB which are altered as described above.

“Amphiphile” refers to a molecule, which consists of a water-soluble (hydrophilic) and an oil-soluble (lipophilic) part. The lipophilic part preferably contains at least one alkyl chain having at least 10, preferably at least 12 carbon atoms.

“Angiogenesis” refers to the formation of new blood vessels. Endothelial cells form new capillaries in vivo when induced to do so, such as during wound repair or in tumor formation or certain other pathological conditions referred to herein as angiogenesis-associated diseases.

“Carrier” refers to a vehicle which is suitable for administering a diagnostic or therapeutic agent. The term also refers to (a) pharmaceutical acceptable component(s) that contain(s), complexes or is otherwise associated with an agent to facilitate the transport of such an agent to its intended target site. Carriers include those known in the art, such as liposomes, polymers, lipid complexes, serum albumin, antibodies, cyclodextrins and dextrans, chelates, or other supramolecular assemblies.

“Cationic” refers to an agent that has a net positive charge or positive zeta potential under the respective environmental conditions. In the present invention, it is referred to environments where the pH is in the range between 3 and 9, preferably between 5 and 8.

“Cationic amphiphile” or “cationic lipid” refers to encompass any amphiphile or lipid which has a positive charge. In the present invention, it is referred to environments where the pH is in the range between 3 and 9, preferably between 5 and 8.

“Cationic liposome” refers to a liposome which is positively charged. In the present invention, it is referred to environments where the pH is in the cationic lipids or amphiphiles themselves or in admixture with other amphiphiles, particularly neutral or anionic lipids.

“Colloidal carriers” refers to particles or molecular aggregates dispersed in a medium in which they are insoluble and have a size between about 5 nm and 5000 nm.

“Colloidal cationic carrier” refers to a colloidal carrier that has a net positive charge or positive zeta potential under the respective environmental conditions. In the present invention, it is referred to environments where the pH is in the range between 3 and 9, preferably between 5 and 8.

“Cryoprotectant” refers to a substance that helps to protect a species from the effect of freezing.

“Derivative” refers to a compound derived from some other compound while maintaining its general structural features. Derivatives may be obtained for example by chemical functionalization or derivatization.

“Diagnostic agent” or “diagnostically active agent” refers to a pharmaceutically acceptable agent that can be used to localize or visualize a target region by various methods of detection. Such agents include those known in the art, such as dyes, fluorescent dyes, gold particles, iron oxide particles and other contrast agents including paramagnetic molecules, X-ray attenuating compounds (for CT and X-ray) contrast agents for ultrasound, magnetic resonance imaging (MRI), X-ray emitting isotopes (scintigraphy), and positron-emitting isotopes (PET).

“Drug” as used herein refers to a pharmaceutically acceptable pharmacologically active substance, physiologically active substances and/or substances for diagnosis use.

“Liposome” refers to a microscopic spherical membrane-enclosed vesicle (about 50-2000 nm diameter) made artificially in the laboratory. The term “liposome” encompasses any compartment enclosed by a lipid bilayer. Liposomes are also referred to as lipid vesicles. In order to form a liposome the lipid molecules comprise elongated nonpolar (hydrophobic) portions and polar (hydrophilic) portions. The hydrophobic and hydrophilic portions of the molecule are preferably positioned at two ends of an elongated molecular structure. When such lipids are dispersed in water they spontaneously form bilayer membranes referred to as lamellae. The lamellae are composed of two monolayer sheets of lipid molecules with their non-polar (hydrophobic) surfaces facing each other and their polar (hydrophilic) surfaces facing the aqueous medium. The membranes formed by the lipids enclose a portion of the aqueous phase in a manner similar to that of a cell membrane enclosing the contents of a cell. Thus, the bilayer of a liposome has similarities to a cell membrane without the protein components present in a cell membrane. As used in connection with the present invention, the term liposome includes multilamellar liposomes, which generally have a diameter in the range of about 1 to about 10 micrometers and are comprised of anywhere from two to hundreds of concentric lipid bilayer alternating with layers of an aqueous phase, and also includes unilamellar vesicles which are comprised of a single lipid bilayer. The latter can be produced by subjecting multilamellar liposomes to ultrasound, by extrusion under pressure through membranes having pores of defined size, or by high pressure homogenization. A further result of these procedures is, that often well defined size distributions of the liposomes are achieved. By extrusion through membranes of defined pore size (typical values are 100, 200, 400 or 800 nm), liposomes with a size distribution close to the pore size of the membrane can be achieved. By ultrasound and high pressure homogenization procedures, defined size distributions are obtained by molecular self-organization as a function of the experimental conditions.

“Liposomal paclitaxel” or “lipid complexed paclitaxel” means a liposomal preparation comprising paclitaxel encapsulated within liposomes. A specific liposomal paclitaxel formulation is EndoTAG®-1. EndoTAG®-1, sometimes also referred to as MBT-0206, is a liposomal paclitaxel with a molar ratio of 50:47:3 mole % of DOTAP, DOPC and paclitaxel. EndoTAG®-1 is a registered trademark in Germany.

“Liposomal preparation” and “liposomes” are used synonymously throughout the present application. The liposomal preparation may be a component of a “pharmaceutical composition” and may be administered together with physiologically acceptable excipients such as a buffer.

“Nanoparticles” refer in the current context to any type of colloidal particle in the size rage between 1 nm and 10000 nm, preferably in the range between 10 nm and 1000 nm.

“Negatively Charged Lipids” refer to lipids that have a negative net charge. In the present invention, it is referred to environments where the pH is in the range between 3 and 9, preferably between 5 and 8. Examples are phosphatidic acid, phosphatidylserine, phosphatidylglycerol, phosphatidylinositol (not limited to a specific sugar), fatty acids, sterols.

“Neutral Lipids” refer to lipids that have a neutral net charge such as cholesterol, 1,2-diacyl-sn-glycero-3-phosphoethanolamine, including but not limited to dioleoyl (DOPE), 1,2-diacyl-glycero-3-phosphocholines, Sphingomyelin. In the present invention, it is referred to environments where the pH is in the range between 3 and 9, preferably between 5 and 8.

“Particle diameter” refers to the size of a particle. To experimentally determine particle diameters, dynamic light scattering (DLS) measurements, for example using a Malvern Zetasizer 1000 or 3000 (Malvern, Herrenberg, Germany) can be performed.

“Targeted delivery” refers to the selective binding, accumulation or uptake of compounds in a certain tissue region. Delivery can be locally confined and/or directed to a certain type of tissue or cells.

“Taxane” refers to the class of antineoplastic agents having a mechanism of microtubule action and having a structure that includes the unusual taxane ring structure and a stereospecific side chain that is required for cytostatic activity. Taxane further refers to a variety of known taxane derivatives, including both hydrophilic derivatives, and hydrophobic derivatives. Taxane derivatives include, but not limited to, galactose and mannose derivatives described in International Patent Application No. WO 99/18113; piperazino and other derivatives described in WO 99/14209; taxane derivatives described in W099/09021, WO 98/22451, and U.S. Pat. No. 5,869,680; 6thio derivatives described in WO 98/28288; sulfenamide derivatives described in U.S. Pat. No. 5,821,263; and paclitaxel derivatives described in U.S. Pat. No. 5,415,869.

“Treatment”, “treating”, “treat” and the like are used herein to generally mean obtaining a desired pharmacologic and/or physiologic effect. The effect may be prophylactic in terms of completely or partially preventing a disease or symptom thereof and/or may be therapeutic in terms of a partial or complete stabilization or cure for a disease and/or adverse effect attributable to the disease. “Treatment” as used herein covers any treatment of a disease in a mammal, particularly a human, and includes: (a) preventing the disease or symptom from occurring in a subject which may be predisposed to the disease or symptom but has not yet been diagnosed as having it; (b) inhibiting the disease symptom, i.e., arresting its development; or (c) relieving the disease symptom, i.e., causing regression of the disease or symptom.

“Therapeutic agent” refers to a species of agents that prevents or reduces the extent of the pathology of a disease such as multiple sclerosis or other diseases disclosed herein.

“Total carrier components” or “total liposomal components” refers to the amount of components that constitute the carrier or the liposomal membranes. The carrier or liposomal components are preferably constituted by the lipids and other amphiphilic or hydrophobic components, including active agents that are bound to or integrated into the carrier, e.g. into the liposomal membrane.

“Zeta potential” refers to measured electrical potential of a colloidal particle in aqueous environment, measured with an instrument such as a Zetasizer 3000 using Laser Doppler micro-electrophoresis under the conditions specified. The zeta potential describes the potential at the boundary between bulk solution and the region of hydrodynamic shear or diffuse layer. The term is synonymous with “electrokinetic potential” because it is the potential of the particles which acts outwardly and is responsible for the particle's electrokinetic behavior.

A first aspect of the present invention is the use of cationic colloidal carriers for the targeted delivery of active compounds to altered sites or inflammatory sites of the BBB and/or BNB vasculature.

A further aspect is the use of a cationic colloidal carrier composition comprising at least one active agent for the preparation of an agent, i.e. a pharmaceutical composition, for the diagnosis or treatment of a neurological inflammatory or degenerative disease.

A further aspect is the use of a cationic colloidal carrier composition comprising at least one active agent for the preparation of an agent, i.e. a pharmaceutical composition, for the diagnosis or treatment of a demyelinating disease, particularly an inflammatory demyelinating disease.

Thus, the current invention discloses a method of treating or diagnosing a neurological inflammatory disease, or a degenerative disease by administering a cationic colloidal carrier composition comprising an active agent to a subject in need thereof, preferably a human patient.

It is another aspect of the current invention to disclose a method of increasing the concentration of an active agent at an altered or inflammatory site of the BBB and/or BNB vasculature in comparison to the concentration of said agent at the un-altered or un-inflamed vasculature by administering said active agent in a cationic colloidal carrier composition.

The group of diseases comprises, but is not restricted to, multiple sclerosis (MS) and other inflammatory neurological diseases in the CNS, Guillain-Barré Syndrome and other inflammatory neurological diseases in the PNS, as well as of their animal models, experimental autoimmune encephalomyelitis and experimental autoimmune neuritis.

Most preferably, the methods of the present invention are used to treat multiple sclerosis, e.g., multiple sclerosis variants such as Neuromyelitis Optica (Decic's Disease), Diffuse Sclerosis, Transitional Sclerosis, Acute Disseminated Encephalomyelitis, and Optic Neuritis, but also Guillain-Barré Syndrome, virus-, bacteria- or parasite-related demyelinating or otherwise degenerative brain disease such as encephalopathies related to HIV, meningococcal or toxoplasma infections, central malaria, Lyme's disease etc.

The present invention also discloses the use of a cationic colloidal carrier composition for the targeted delivery of an active agent to an inflammatory site or an activated vascular site for the diagnostic application in multiple sclerosis.

In a preferred embodiment, the cationic colloidal carrier is a colloidal carrier particle selected from the group comprising a liposome, a solid lipid particle, a solid drug particle, a polymer or polymer particle, a solid gold or metal particle, a quantum dot, a dendrimer, a fullerene, a carbon nanotube, a polymer capsule, or any other nanoparticle in the size range between about 1 and about 5000 nm. More preferably the size of the colloidal carrier particle is between 10 and 1000 nm.

It is another aspect of the present invention that the cationic colloidal carrier has a zeta potential in the range of about +20 mV to 100 mV, preferably at least about +30 mV in about 0.05 mM KCl solution at about pH 7.5 at room temperature.

Cationic colloidal carriers can be manufactured by mixing cationic components to the particle forming moieties, for example by inserting cationic amphiphiles to a liposome, emulsion droplet, micelle, or solid lipid particle. Cationic colloidal carriers can also be manufactured by chemical functionalization of the particle with cationic moieties, or by physisorption or self-assembly processes, for example by binding cationic polyelectrolytes to nanoparticles. Furthermore, drug nanoparticles or cationized gold particles can be used as cationic colloidal carriers. Furthermore cationic polymers can be used.

In the most preferred embodiment of the invention, the cationic colloidal carrier is a cationic liposomal preparation.

Cationic lipids for formation of cationic carriers, e.g. liposomes, preferably consist of a cationic hydrophilic head group and a hydrophobic moiety which can be formed from one, two or more acyl chains. The chains can be of different length, they can be saturated or (poly) unsaturated. The chains can be linear or branched.

In a preferred embodiment, the liposomal preparation of the present invention comprises a cationic lipid or a mixture of cationic lipids in an amount of at least about 30 mol %, more preferably at least about 50 mol % of total liposomal components.

Preferred cationic lipids of the liposomal preparation are N-[1-(2,3-diacyloxy)propyl]-N,N,N-trimethyl ammonium salts, e.g. the methylsulfate or the chloride salts. Preferred representatives of the family of -TAP lipids are DOTAP (dioleoyl-), DOTAP (dimyristoyl-), DPTAP (dipalmitoyl-), or DSTAP (distearoyl-). Other useful lipids for the present invention may include:

DDAB, dimethyldioctadecyl ammonium bromide and analogues thereof; N-[1-(2,3-dioleoyloxy)propyl]-N,N-dimethyl amine (DODAP); 1,2-diacyloxy-3-dimethylammonium propanes, (including but not limited to: dioleoyl, dimyristoyl, dilauroyl, dipalmitoyl and distearoyl; also two different acyl chain can be linked to the glycerol backbone); N-[1-(2,3-dioleyloxy)propyl]-N,N,N-trimethylammonium chloride (DOTMA); 1,2-dialkyloxy-3-dimethylammonium propanes, (including but not limited to: dioleyl, dimyristyl, dilauryl, dipalmityl and distearyl; also two different alkyl chain can be linked to the glycerol backbone); dioctadecylamidoglycylspermine (DOGS); 3β-[N—(N′, N′-dimethylamino-ethane)carbamoyl]cholesterol (DC-Chol); 2,3-dioleoyloxy-N-(2-(sperminecarboxamido)-ethyl)-N,N-dimethyl-1-propanaminium trifluoro-acetate (DOSPA); β-alanyl cholesterol; cetyl trimethyl ammonium bromide (CTAB); diC14-amidine; N-tert-butyl-N′-tetradecyl-3-tetradecylamino-propionamidine; 14Dea2; N-(alpha-trimethylammonioacetyl)didodecyl-D-glutamate chloride (TMAG); O,O′-ditetradecanoyl-N-(trimethylammonio-acetyl)diethanolamine chloride; 1,3-dioleoyloxy-2-(6-carboxy-spermyl)-propylamide (DOSPER); N,N,N′,N′-tetramethyl-N,N′-bis(2-hydroxylethyl)-2,3-dioleoyloxy-1,4-butanediammonium iodide; 1-[2-(acyloxy)ethyl]2-alkyl(alkenyl)-3-(2-hydroxyethyl)-imidazolinium chloride derivatives as described by Solodin et al. (Solodin et al., 1995), such as 1-[2-(9(Z)-octadecenoyloxy)ethyl]-2-(8(Z)-heptadecenyl-3-(2-hydroxyethyl)imidazolinium chloride (DOTIM), 1-[2-(hexadecanoyloxy)ethyl]-2-pentadecyl-3-(2-hydroxyethyl)imidazolinium chloride (DPTIM), 2,3-dialkyloxypropyl quaternary ammonium compound derivatives, containing a hydroxyalkyl moiety on the quaternary amine, as described e.g. by Felgner et al. (Felgner et al., 1994) such as: 1,2-dioleoyl-3-dimethyl-hydroxyethyl ammonium bromide (DORI), 1,2-dioleyloxypropyl-3-dimethyl-hydroxyethyl ammonium bromide (DORIE), 1,2-dioleyloxypropyl-3-dimethyl-hydroxypropyl ammonium bromide (DORIE-HP), 1,2-dioleyloxypropyl-3-dimethyl-hydroxybutyl ammonium bromide (DORIE-HB), 1,2-dioleyloxypropyl-3-dimethyl-hydroxypentyl ammonium bromide (DORIE-Hpe), 1,2-dimyristyloxypropyl-3-dimethyl-hydroxylethyl ammonium bromide (DMRIE), 1,2-dipalmityloxypropyl-3-dimethyl-hydroxyethyl ammonium bromide (DPRIE), 1,2-disteryloxypropyl-3-dimethyl-hydroxyethyl ammonium bromide (DSRIE); cationic esters of acyl carnitines as reported by Santaniello et al. [U.S. Pat. No. 5,498,633]; cationic triesters of phosphatidylcholine, i.e. 1,2-diacyl-sn-glycerol-3-ethylphosphocholines, where the hydrocarbon chains can be saturated or unsaturated and branched or non-branched with a chain length selected from the group consisting of C₁₂, C₁₃, C₁₄, C₁₅, C₁₆, C₁₇, C₁₈, C₁₉, C₂₀, C₂₁, C₂₂, C₂₃, and C₂₄, the two acyl chains being not necessarily identical.

In a preferred embodiment, the liposomal preparation comprises at least one neutral and/or anionic lipid. Neutral lipids are lipids which have a neutral net charge. Anionic lipids or amphiphiles are molecules which have a negative net charge. These can be selected from sterols or lipids such as cholesterol (Chol), phospholipids, lysolipids, lysophospholipids, sphingolipids or pegylated lipids with a neutral or negative net change. Useful neutral and anionic lipids thereby include: phosphatidylserine, phosphatidylglycerol, phosphatidylinositol (not limited to a specific sugar), fatty acids, sterols, containing a carboxylic acid group for example, cholesterol, 1,2-diacyl-sn-glycero-3-phosphoethanolamines, including, but not limited to, 1,2-dioleyl-sn-glycero-3-phosphoethanolamine (DOPE), 1,2-diacyl-glycero-3-phosphocholines, including, but not limited to 1,2-dioleyl-sn-glycero-3-phosphocholine (DOPC), and sphingomyelin. The fatty acids linked to the glycerol backbone are not limited to a specific length or number of double bonds. They may be linear or branched. Phospholipids may also contain two different fatty chains. Neutral or anionic lipids may also be used as conjugates with polyalkyleneoxides, e.g. polyethyleneglycol (PEG). Preferably the further lipids are in the liquid crystalline state at room temperature and they are miscible (i.e. a uniform phase can be formed and no phase separation or domain formation occurs) with the cationic lipid, in the ratio as they are applied. In a preferred embodiment the neutral or anionic lipid is DOPC or DOPE or PEG conjugates thereof such as DOPE-PEG.

In a further preferred embodiment, the liposomal preparation comprises optionally neutral and/or anionic lipids, preferably DOPC in an amount of about up to about 70 mole %, preferably up to about 55 mole %, more preferably from about 45 mole % to about 55 mole % of total liposomal components.

The liposomal preparations of the present invention can be obtained by homogenizing the hydrophobic compounds in water by a suitable method and further processing. Homogenizing can be obtained by mechanical mixing, stirring, high-pressure homogenization, adding an organic phase comprising the hydrophobic compounds to the aqueous phase, spraying techniques, supercritical fluid technology or any other technique suitable in order to obtain lipid dispersions in water.

In a preferential embodiment, the liposomal preparations of the present invention can be obtained by methods like the “film method” or by organic solvent (e.g. ethanol) injection, which are known to those skilled in the art (WO 2004/002468).

The cationic colloidal carrier preparation can be dehydrated, stored for extended periods of time while dehydrated, and then rehydrated when and where it is to be used, without losing a substantial portion of its contents during the dehydration, storage and rehydration processes. To achieve the latter, one or more protective agents, such as cryoprotectants, may be present. Thus, the inventive cationic liposome preparation preferably comprises a cryoprotectant, wherein the cryoprotectant is selected from a sugar or an alcohol or a combination thereof. Preferably, the cryoprotectant is selected from trehalose, maltose, sucrose, glucose, lactose, dextran, mannitol or sorbitol.

In a further preferred embodiment, the carrier preparation comprises trehalose in the range of about 5% (m/v) to about 15% (m/v) with respect to the total volume of the preparation.

In a further preferred embodiment, the carrier preparation comprises glucose in the range of about 2.5% (m/v) to about 7.5% (m/v) with respect to the total volume of the preparation.

The formulation of the cationic liposomes of the present invention may vary. In a preferred embodiment the molar ratio is 50:47:3 of DOTAP, DOPC and paclitaxel. This formulation is also designated MBT-0206 or EndoTAG®-1.

Liposomes of various sizes are useful in the present invention. In a preferred embodiment of the present invention cationic liposomes have an average particle diameter from about 25 nm to about 500 nm, preferably from about 50 to about 500 nm, more preferably from about 100 nm to about 300 nm.

The cationic colloidal composition comprises an active agent. The active agent can be hydrophilic (water soluble), hydrophobic or amphiphilic. It can be a small molecule (molecular weight up to an order of 1 kDa) or it can be a polymer, a polypeptide, a protein or nucleic acid. Nucleic acids as active agents may be selected from DNA, RNA, iRNA, and preferably siRNA. The active agent can be a therapeutic or diagnostic agent or a combination of a therapeutic agent and a diagnostic agent.

For therapeutic purposes, the active agent may be an inhibitor of angiogenesis, an activator of angiogenesis, an immunomodulatory agent, an immunosuppression agent, an anti-inflammatory agent, a cell-adhesion inhibitor, an anti-oxidant or any combination thereof.

The active agent can be selected from a cytotoxic or cytostatic substance such as an anti-tumor or an anti-endothelial cell active substance, a chemotherapeutic agent or an immunological active substance. In a more preferred embodiment, the active agent is selected from a taxane, a camptothecin, a statin, a depsipeptide, thalidomide, other agents interacting with microtubuli such as discodermolide, laulimalide, isolaulimalide, eleutherobin, Sarcodictyin A and B, and in a most preferred embodiment, it is selected from paclitaxel, docetaxel, camptothecin or any derivative thereof.

In a preferred embodiment, the composition comprises paclitaxel in an amount of at least about 2 mole % to about 8 mole %, preferably from at least 2.5 mole % to about 3.5 mole % of total liposomal components. The cationic colloidal composition of the present invention comprises substantially no crystalline paclitaxel.

The active agent can also be selected from immunomodulatory cytostatic substances like azathioprine, mycophenolate mofetil, cyclophosphamide, mitoxantrone, methotrexate, linomide derivative (Laquinimod), pixantrone, and Cyclosporin A.

The active agent can also be a cytokine or a proinflammatory cytokine inhibitor such as IFN β-1a, IFN β-1b, interferon-α, interferon-tau, tumor necrosis factor (TNF) inhibitors (for example etanercept, infliximab and adalimumab), or antibodies against proinflammatory cytokines. In a more preferred embodiment IFN β, a derivative thereof, or a functional fragment thereof is used.

Other examples of active agents are immunosuppressive antibodies (e.g. anti-CD3, anti CD4, anti-CD52, anti-IL2 receptor or anti-CD20 antibodies) or agents that are directed at cell adhesion and costimulatory molecules like anti-CD11/CD8 antibodies, small molecule inhibitors of integrins or antibodies against α4 integrin, CD54, CD2, CD58, CD154 or CD45. In a preferred embodiment, anti-α4 integrin antibodies are used as an active agent. It is another preferred embodiment of the invention to use peptides as active agents that target the immune system by MHC binding. Glatiramer acetate (Copaxone®) is a preferred example of these species.

Corticosteroids, preferably prednisolone or dexamethasone, might also be used as active agents in the current invention.

Further, an enzyme inhibitor may be used as an active compound. For example, protease inhibitors, e.g. indinavir (IDV), MMP inhibitors, e.g. minocycline or reverse transcriptase inhibitors, e.g. zidovudine (AZT) are used.

Furthermore, anti-oxidants such as PUFA, Vitamin E, lipoic acid, N-acetylcysteine and Vitamin B12 may be used as active compounds. For example, the cationic colloidal carrier can comprise two omega-3 polyunsaturated fatty acids (PUFA) such as eicosapentanoic acid (EPA, C20:5) and docoexaenoic acid (DHA, C22:6) and Vitamin E as antioxidants.

Alternatively, the active agent may be fumarate, fingolimod (FTY-720), mycophenolic acid, cladribine, teriflunomid or a derivative of said compounds.

The compositions of the present invention can be administered systemically, preferably intravenously. Preferably, the compositions are administered to a mammal, e.g. a human patient.

Prior to administration, the formulation may be reconstituted in an aqueous solution in the event that the formulation was freeze dried. The required application volume is calculated from the patient's body weight and the dose schedule.

The cationic carrier compositions of the present invention may be used to treat any form of neuroinflammatory, neurodegenerative or demyelinating disease such as MS or other neurological disease involving BBB and/or BNP disruption at inflammation sites. The pharmaceutical composition of the present invention is particularly advantageous in treating MS in human patients because the colloidal carrier is safe and able to deliver active agents directly at the site of inflammation at the level of the BBB protecting it from deterioration. This can in turn allow to use lower doses of a previously used active agent.

The cationic colloidal composition of the invention may be administered as a first line treatment or as a second or third line treatment. Further, the composition may be administered as a monotherapy or as a combination therapy with further active agents such as e.g. interferons.

The combination therapy may be simultaneous, separate, or sequential combination therapy with a jointly effective dose of at least one further active agent and/or heat and/or radiation and/or cryotherapy. The further active agent may be comprised in the same or a different cationic colloidal composition or may be administered in a different non-cationic composition.

The at least one further active agent may be a cytotoxic or cytostatic substance as described above, such as an anti-endothelial cell active substance, an immunological active substance, a compound that reduces or a substance which eliminates hypersensitivity reactions. Further, it is preferred that the active agent and the further active agents are different.

It is another embodiment of the disclosed invention to use a diagnostic agent as an active agent in the disclosed compositions for the targeting to an inflammatory site or an altered site of the BBB vasculature. These compositions can be used for the diagnosis, e.g. of an inflammatory demyelination disease.

The diagnostic or imaging label may be selected from a group comprising metal ions or metal ion chelates (preferably chelates from transition metals such as gadolinium, lutetium, or europium) for example as used for MRI and X-ray contrast are used. In a more preferred embodiment gadolinium chelates are used as active agents.

Furthermore, the imaging label may be selected from the group comprising of fluorescent labels, histochemical labels, immunohistochemical labels, or radioactive labels. Preferred radioactive labels are inter alia isotopes of iodine, indium, gallium, ruthenium, mercury, rhenium, tellurium, thulium, and more preferably technetium.

It should be noted that all preferred embodiments discussed for one or several aspects of the invention also relate to all other aspects. This particularly refers to the amount and type of cationic lipid, the amount and type of neutral and/or anionic lipid, the amount and type of active agent, the amount and type of further active agent for combination therapy, and the type of disorder to be treated.

The following examples should be illustrative only but are not meant to be limiting to the scope of the invention. Other generic and specific configurations will be apparent to those skilled in the art.

### FIGURE LEGENDS

FIG. 1: Cryosection images of spinal cord with confocal microscopy after injection of LipoRed. Spinal cord was resected, fixed in 4% paraformaldehyde in 120 mM phosphate buffer pH 7.4, and OCT embedded.

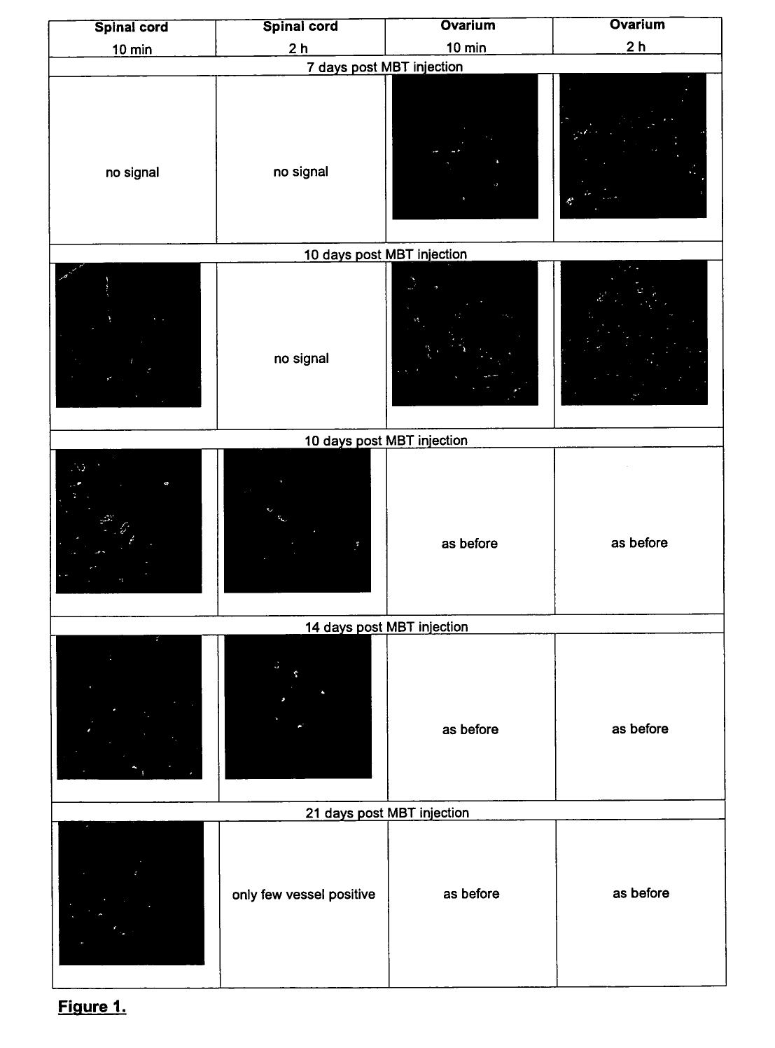

### EXAMPLES

### 1. Production of Cationic Colloidal Carrier Compositions

### 1.1 Preparation of Cationic Liposomes Comprising a Hydrophobic Compound, e.g. Paclitaxel

The production of cationic liposomes comprising a hydrophobic cytotoxic agent, e.g. paclitaxel can be performed by standard procedures for manufacturing of liposomes, for example as described in WO 2004/002468. Usually, the hydrophobic drug is mixed with the lipids and dispersed in a suitable way in the aqueous phase. A preparation procedure using the so-called film method is described in the literature (Krasnici et al., 2003). A further procedure which is particularly suitable for large scale production is the ‘ethanol injection’ method. Briefly, the production scheme can be summarized as follows: Multilamellar liposomes are produced by injection of an ethanol solution comprising the lipids and the hydrophobic drug under stirring into the aqueous phase (ethanol injection). A suitable composition of the liposomes, e.g. DOTAP/DOPC/paclitaxel in a molar ratio 50/47/3, with a total final concentration in water of 10 mM. For the ethanol solution, an appropriate concentration of the lipid fraction is 400 mM. The size distribution of the polydisperse liposome preparation is adjusted by extrusion across membranes of e.g. 200 nm pore size (Osmonics, Minnetonka, Minn., USA) with a pressure of about 5-7 bar. The resulting suspension of liposomes with defined sized distribution may be sterile filtrated e.g. across a Durapore membrane filter of 220 nm pore size (Millipore, Molsheim, France). By lyophilization of the resulting sterile liposome product a shelf life of more than 18 months can be obtained. The liposomal preparation which is obtained from the method described here is denoted as well as EndoTAG®-1.

These methods are also suitable for production of liposomal preparations comprising other hydrophobic active agents as described in the present patent application.

### 1.2. Preparation of Cationic Liposomes Comprising a Water-Soluble Compound

### 1.2.1 Preparation of Cationic Liposomes Comprising Gadolinium

The production of liposomes comprising a water-soluble compound, e.g. the contrast agent Gadovist®, a gadolinium chelate, is described. Gadolinium-loaded liposomes can be used as contrast agent for MRI and X-ray imaging and for therapeutic purposes.

The method is also suitable for the preparation of liposomal products from other types of water-soluble compounds in the context of the present patent application.

Here; as examples the production of liposomal preparations with a composition of the lipid membrane

### DSTAP/DMPC/Chol/DOPE-PEG 30/20/45/5 (mol %)

### DSTAP/DMTAP/Chol/DOPE-PEG 30/20/45/5 (mol %)

### DSTAP/DOTAP/Chol/DOPE-PEG 30/20/45/5 (mol %)

are described. The method is applicable also for preparation of liposomes with another composition.

### Methods

Liposome preparation was performed by the ‘film method’ with subsequent extrusion. The necessary amounts of lipid components for the above given molar compositions in the aqueous phase were dissolved in about 25 ml of chloroform and added to a 250 ml round bottom flask. The solvent was evaporated at bath temperature of about 60° C., by applying 150 mbar for 15 minutes and, subsequently, 10 mbar for one hour. The resulting lipid film was rehydrated at 60° C. with 6 ml of a solution of Gadovist® (1000 mM) comprising 5% glucose by gently swiveling the flask.

The obtained preparation of multilamellar, polydisperse liposomes was extruded (pressure 6-7 bar), through a membrane of 800 nm pore size (1×), a membrane of 400 nm pore size (1×), and a membrane of 200 nm pore size (3×). Excess, non encapsulated gadolinium was removed by dialysis against an aqueous phase comprising 5% glucose. A cellulose membrane with 8-10 kDa pore size was used. The medium was exchanged 4 times every 9-15 hours. The volume of the liposome preparation was measured before and after dialysis in order to determine volume changes (Liposome preparation was performed at a high concentration in order to take account for dilution effects due to dialysis).

For size measurements, photon correlation spectroscopy (PCS) measurements were performed, using a Malvern Zetasizer 1000. The preparations were diluted to a total lipid concentration of 1 mM.

The amount of encapsulated Gd was determined by ICP/MS (Inductively Coupled Plasma-Mass Spectrometry) measurements. Further, the Zeta potential (Zₐᵥₑ) and the polydispersity index (PI, ISO 13320) were determined. For these measurements, the preparations were diluted with ethanol and HNO₃ to a Gd concentration of about 1 mg/l. Results for are shown in Table 1.

TABLE 1

| | Total lipid | Gadolinium | | |
|---|---|---|---|---|
| | concentration | concentration | Zₐᵥₑ | |
| Formulation | (mM) | (mM) | (nm) | PI |
| DSTAP/DMPC/Chol/ | 30 | 39 | 198 | 0.07 |
| DOPE-PEG | | | | |
| 30/20/45/5 mol % | | | | |
| DSTAP/DMTAP/Chol/ | 32 | 48 | 206 | 0.14 |
| DOPE-PEG | | | | |
| 30/20/45/5 mol % | | | | |
| DSTAP/DOTAP/Chol/ | 31 | 39 | 196 | 0.09 |
| DOPE-PEG | | | | |
| 30/20/45/5 mol % | | | | |

### 1.2.2. Preparation of Cationic Liposomal Methotrexate (MTX) Preparations

### Preparation of Endo-MTX Formulations by Co-Extrusion

20 mM DOTAP liposomes (20 ml) were prepared by the lipid film method as described in WO 2004002468 by Mundus et al. and rehydration was performed with 10% trehalose. Liposomes were subsequently mixed with 20 ml of a sodium MTX solution (2.2 mM, prepared from diluting a 220 mM sodium MTX solution with 10% trehalose). The resulting solution (theoretical concentration 10 mM DOTAP and 1.1 mM MTX) was extruded 5 times through a polycarbonate membrane with 200 nm pore size. Subsequently, HPLC and PCS analytics were performed. The results are as follows:

### DOTAP: 8.4 mM

MTX 1.14 mM (for HPLC methods, see below)

### Zave=156 nm

### PI 0.29

Zeta potential: +59.3 mV.

MTX release from liposomes was determined by centrifugation through a Centricon tube (MWCO=30,000, 4500 rcf, 180 min) and was found to be 1.4% of the MTX concentration. The formulation is stable at 4° C. for at least 16 weeks.

### Preparation of PEGylated Endo-MTX Formulations by Co-Extrusion

20 mM DOTAP/PEG-DOPE liposomes with a molar ratio 95/5 mol % (total volume of 20 ml) were prepared by the lipid film method as described in WO 2004002468 by Mundus et al. and rehydration was performed with 10% trehalose. Liposomes were subsequently mixed with 20 ml of a sodium MTX solution (2.2 mM, prepared from diluting a 220 mM sodium MTX solution with 10% trehalose). The resulting solution (theoretical concentration 9.5 mM DOTAP, 0.5 mM PEG7DOPE, 1.1 mM MTX) was extruded 5 times through a polycarbonate membrane with a pore size of 200 nm. Subsequently, HPLC and PCS analytics were performed. The results are as follows:

### DOTAP: 8.83 mM

MTX 1.02 mM (for HPLC methods, see below)

### Zave=161 nm

### PI 0.225

Zeta potential: −2.4 mV (+0.5 mV after 1:10 dilution in a solution containing 50 mM KCl and 10% trehalose).

MTX release from liposomes was determined by centrifugation through a Centricon tube (MWCO=30,000, 4500 rcf, 180 min) and was found to be 2.7% of the MTX concentration. The formulation is stable at 4° C. for at least 16 weeks.

### Preparation of Endo-MTX Formulations by Mixing (MRa0036)

20 mM DOTAP liposomes (20 ml) were prepared by the lipid film method as described in WO 2004002468 by Mundus et al. and rehydration was performed with 10% trehalose. Then, the liposomes were extruded 5 times through 200 nm membrane (polycarbonate). The resulting SUV suspension was mixed with 20 ml of a sodium MTX solution (2.2 mM, prepared from diluting a 220 mM sodium MTX solution with 10% trehalose). The resulting solution had 10 mM DOTAP and 1.1 mM MTX. Subsequently, HPLC and PCS analytics were performed. The results are as follows:

### DOTAP: 9.6 mM

MTX 1.14 mM (for HPLC methods, see below)

### Zave=145 nm

### PI 0.373

Zeta potential: +50 mV.

MTX release from liposomes was determined by centrifugation through a Centricon tube (MWCO=30,000, 4500 rcf, 180 min) and was found to be 1.4% of the MTX concentration. The formulation is stable at 4° C. for at least 16 weeks.

### Preparation of PEGylated Endo-MTX Formulations by Mixing

20 mM DOTAP/PEG-DOPE liposomes with a molar ratio 95/5 mol % (total volume of 20 ml) were prepared by the lipid film method as described in WO 2004002468 by Mundus et al. and rehydration was performed with 10% trehalose. The liposomes were subsequently extruded 5 times through 200 nm membrane (polycarbonate). Then, the resulting SUVs were mixed with 20 ml of a sodium MTX solution (2.2 mM, prepared from diluting a 220 mM sodium MTX solution with 10% trehalose), resulting in a suspension with 9.5 mM DOTAP, 0.5 mM PEG-DOPE, 1.1 mM MTX. Subsequently, HPLC and PCS analytics were performed. The results are as follows:

### DOTAP: 9.6 mM

MTX 1.14 mM (for HPLC methods, see below)

### Zave=157 nm, PI 0.259

Zeta potential: 1.1 mV (1.1 mV (after 1:10 dilution in a solution containing 50 mM KCl and 10% trehalose).

MTX release from liposome was determined by centrifugation through Centricon tube (MWCO=30,000, 4500 rcf, 180 min) and was found to be 3.6% of the MTX concentration. The formulation is stable at 4° C. for at least 16 weeks.

Analysis of the DOTAP content by HPLC

As stationary phase, a C8 column Luna 5μ C8 (2) 100 Å, 150×2 mm (Phenomenex) is used. The mobile phase is composed of water with 0.1% TFA (solvent A) and acetonitrile with 0.1% TFA (solvent B), the following gradient program is run:

| Time (min) | Solv. B (%) |
|---|---|
| 0.00 | 50. |
| 4.12 | 50 |
| 7.06 | 75 |
| 14.13 | 100 |
| 21.20 | 100 |
| 23.56 | 50 |
| 30.00 | 50 |

Column temperature: 45° C.
Injection volume: 5 μl
Wavelength for detection: 205 nm
Run time: 30 min

### Analysis of the Methotrexate Content by HPLC

An isocratic method is employed, using a C18 stationary phase (Luna 5μ C18 (2) 100 Å, 150×2 mm (Phenomenex). The mobile phase is composed of 10 mM NH4OAc pH: 6.0 and acetonitrile at a ratio 93/7 (v/v).

Column temperature: 40° C.
Injection volume: 10 μl
Wavelength for detection: 310 nm
Run time: 15 min

### 1.3 Preparation of Liposomes Comprising a Compound Capable of Interacting with Molecules, e.g. Camptothecin

The production of liposomes comprising an active compound which displays interactions with the cationic lipid matrix, e.g. a compound with at least one negatively charged group, is described. Here, as an example, the production of liposomes comprising the topoisomerase inhibitor camptothecin is described.

In principle any of the numerous methods for liposome manufacturing as described in the art is suitable. Here, a particularly simple and efficient method is described, which avoids the use of organic solvent. Liposomes are produced by simple stirring, and the drug is loaded to the liposomes by adding a suitable solution to the empty liposomes.

### Method

157.2 mg of DOTAP were stirred in 15 ml 9% trehalose with a magnetic stirrer for 15 hours. An opalescent suspension was obtained, free of particles as visible by the eye. The aqueous phase was stirred with a magnetic stirrer at slow-medium speed for about 1 hour. Camptothecin (CPT)-carboxylate solution (37.5 mM) was added either before (a) or after extrusion (b). 80 μl of CPT-carboxylate solution were added to 4 ml of the suspensions.

The resulting particles were extruded through polycarbonate membranes of 200 nm pore size at 5 bar. Zeta potential and polydispersity index were determined. These parameters were not affected by adding the drug to the liposomes (Table 2). The fraction of free CPT was very low, independently if the drug was added before or after extrusion (Table 3).

TABLE 2
size measurements

| | Z_{Ave} | |
|---|---|---|
| Sample | (nm) | PI |
| DOTAP | 164 | 0.31 |
| DOTAP/CPT, CPT added before extrusion | 165 | 0.22 |
| DOTAP/CPT, CPT added after extrusion | 164 | 0.24 |

TABLE 3
fraction of free camptothecin

| | time after | fraction of |
|---|---|---|
| | mixing/extrusion | free CPT |
| Sample | (hrs) | (%) |
| DOTAP/CPT, CPT added before extrusion | 0 | 0.6 |
| DOTAP/CPT, CPT added after extrusion | 0 | 0.5 |

Another active compound which can be loaded to cationic carriers by such a procedure is for example methotrexate.

### 1.4 Preparation of Liposomes Comprising an Amphiphilic Compound (LipoRed®)

The production of liposomes comprising an active compound with amphiphilic properties is described. Here, as an example, the production of liposomes comprising a rhodamine labeled lipid is given.

Another active compounds which can be loaded to cationic carriers by such a production scheme are for example PUFAs.

### Materials and Methods

DOTAP-CI, DOPC and Rh-DOPE were obtained from Avanti Polar Lipids (Alabaster, Ala., USA). 5% glucose solution in water for injection use from Braun, Germany was used. Ethanol, p.a. grade was from Merck. Extrusion was performed with an Extruder from Sartorius (Surrey, UK) using polycarbonate membranes with 100 nm pore size (Osmonics, Minnetonka, Minn., USA).

Liposome size was determined by photon correlation spectroscopy, using a Malvern Zetasizer 3000. The particle size distribution was expressed as Z(average), Zₐᵥₑ, and polydisperity index, PI, (ISO 13320).

### Method and Manufacturing Process

Briefly, 773.9 mg DOTAP, 7429.3 mg DOPC and 1367.4 mg Rho-DOPE (rhodamine-labelled DOPE) were added to a calibrated 100 ml flask, and the flask was filled to 100 ml total volume with ethanol.

95.3 ml of the ethanolic stock solution was injected by a pump system under vigorous stirring into 1937.7 g of a 5% glucose solution. The total time of injection was 383 min. After the end of injection the suspension was stirred for one hour.

Subsequently, diafiltration was performed to remove the ethanol from the suspension. 15 runs of filtration with a Sartorius Sartoflow alpha (Sartorius, Surrey UK) were performed. The resulting solution had a slightly higher concentration with respect to the starting conditions (loss of water across the membrane).

The resulting ethanol-free suspension of multilamellar liposomes was extruded 20 times through at PVPF membrane of 100 nm pore size (Polycarbonate, Osmonics, USA) at a pressure of 6-7 bar and at room temperature in an extruder for 2 l total volume (Sartorius, Germany).

After extrusion the lipid concentration (determined by HPLC) after extrusion was 11.4 mM. 255 ml of 5% glucose solution were added to adjust to the theoretical value of 10 mM.

The resulting suspension of monodisperse and unilamellar liposomes was sterile filtrated across a membrane filter of 220 nm pore size (Durapore, Millipore, Monsheim, France).

The liposome suspension was aliquoted in glass vials. After covering the suspensions with argon the vials were sealed with gas-tight taps.

The lipid composition of the formulation was controlled by HPLC analysis, the concentration of Rho-DOPE was in addition determined by fluorescence spectroscopy. The size distribution of the liposomes was determined by photon correlation spectroscopy (PCS).

### Results

Lipid composition as determined by HPLC analysis.

| | | |
|---|---|---|
| DOTAP: | 4.6 | mM |
| DOPC: | 4.4 | mM |
| Rh-DOPE: | 0.50 | mM |
| Osmolarity | 298 | mOsmol/kg |
| Zₐᵥₑ: | 150 | nm |

| | |
|---|---|
| PI: | 0.282 |
| Zeta Potential: | approx. +60 mV |

### 1.5 Preparation of Polymer Based Carrier Particles

Carrier particles can be produced from charged polymers (polyelectrolytes) by different types of self-assembly processes as described in the art (Decher, 1997). Particles can be functionalized by adsorption of cationic polyelectrolytes (Zahr et al., 2005). If necessary, sequential adsorption of positively and negatively charged polyelectrolytes is performed. Particles can be formed in a single step on the basis of chitosan and other polymers by methods such as described in the art (Lee et al., 2006).

### 2. Localization of Rhodamine-Loaded Cationic Liposomes (LipoRed®) in Rat Acute EAE

### Purpose of the Study

The study was performed in order to establish in a model of acute Experimental Allergic Encephalomyelitis (EAE) the occurrence and extent of rhodamine-loaded EndoTAG® (LipoRed®) localization in the spinal cord.

### Materials And Methods

### Test Method

### Clinical Score Assessment

The severity of the clinical signs of EAE was assessed by two independent examiners. Where there was disagreement, in order to reach a consensus a further evaluation was performed by a third examiner who was unaware of the scores as assessed by the two first-line examiners. The severity of EAE was assessed according to a scale ranging from 0 to 5 as follows: 0=normal; 1=limp tail; 2=mild paraparesis; 3=paraplegia; 4=quadriplegia; 5=moribund or death.

### Sacrifice

At the end of the experiment, according to the experimental protocol, selected animals were sacrificed by means of CO₂ inhalation and used for biological sampling.

### Pathological Examination

At the sacrifice the spinal cord was removed from EAE and healthy rats (negative controls), fixed in 4% paraformaldehyde and embedded in Tissue-Tec® O.T.C. Compound for cryopreservation. For the histological examination, 8 μm-thick cryosections were stained with hematoxilin-eosin, while confocal laser microscopy examination was performed on unstained sections. From EAE rats ovary specimens were also obtained, fixed in 4% paraformaldehyde, embedded in OCT and cryosections were used as reference positive control.

### Test and Reference Item/Vehicle

| | |
|---|---|
| Sample | LipoRed ® |
| Aliquots | 10 × 15 ml |
| Description | Rhodamine labelled cationic liposomes |
| Size | Zave (average size): 135 nm; PI = 0.27 |

Storage: 4° C., protected from light

| Composition | Concentration mg/ml | Concentration mmol/L |
|---|---|---|
| DOTAP | 3.59 | 5.15 |
| DOPC | 3.77 | 4.80 |
| Rho-DOPE | 0.79 | 0.604 |
| Glucose | 5015 | |
| Water | 959.1 | |
| Total lipid | 8.14 | 10.55 |

### Administration:

5 mg total lipid/kg body weight iv as slow bolus into the tail vein.
Injection volume at 5 mg/kg: 0.617 μl/g

### Experimental Animals

| | |
|---|---|
| Animal species and strain: | Female Lewis rats |
| Breeder/supplier: | Harlan Italy (Correzzana, Italy) |
| Number of animals in study: | 40 |
| Reserve animals: | 2 |
| Age: | 9-10 weeks |

Animals were subjected to a physical examination (health check) shortly after arrival. Two reserve animals were examined during the pretest period for possible animal exchange.

### Study Design and Animal Allocation

Number of animals/group as follows in Table 4-1:

Rats were immunized by (subcutaneous inoculation into both hind limb foot-pads of 50 μg of guinea pig myelin basic protein in 100 μl complete Freund's adjuvant with 3 mg/ml of inactivated Mycobacterium tuberculosis purchased by Difco Laboratories, Detroit, Mich.).

### Study Design

| | |
|---|---|
| Pretest period: | 7 days |
| Duration after immunization: | 21 days |

Table 4-2: Study schedule

| Major activities | Study day/week/month, time point |
|---|---|
| Mortality | Daily |
| Clinical signs | Daily |
| Clinical scoring | days 7, 10, 12, 14, 17, 21 pi |
| Tissue sampling | At the scheduled time points |
| | (days 7, 10, 12, 14, 17, 21 pi) |

### Sampling and Histological Processing of Organs/Tissues

At sacrifice spinal cord and ovary specimens were obtained as follows in Table 4-3.

TABLE 4-3
Sacrifice plan

| | Day pi | | | | | | Healthy |
|---|---|---|---|---|---|---|---|
| | 7 | 10 | 12 | 14 | 17 | 21 | controls |
| Total number of | 4 | 6 | 8 | 8 | 4 | 4 rats | 6 rats |
| rats | rats | rats | rats | rats | rats | | |
| Time after | | | | | | | |
| LipoRed ® | | | | | | | |
| injection | | | | | | | |
| 10 minutes | 2 | 3 | 4 | 4 | 2 | 2 rats | 1 rat at |
| | rats | rats | rats | rats | rats | | each time |
| | | | | | | | point |
| 2 hours | 2 | 3 | 4 | 4 | 2 | 2 rats | — |
| | rats | rats | rats | rats | rats | | |

### Microscopic Examination and Peer Review

Confocal laser microscopy within 24 hours from sacrifice, hematoxilin-eosin stained sections at the end of the treatment period

### Results

### In-Life Examinations

### Mortality

No mortality was observed in the experiment

### Clinical Observations

The administration of the test compound was well-tolerated

### Clinical Scoring

The summary of the clinical EAE scores observed during the study are reported in Table 5-1.

TABLE 5-1
Clinical scoring at each time point

Days after immunization
(pi)

| | 7 | 10 | 12 | 14 | 17 | 21 |
|---|---|---|---|---|---|---|
| Clinical score | 0 | 1 | 2 | 3 | 3 | 0 |
| (median) | | | | | | |

### Pathology

### Inflammatory Infiltrate

The extent of inflammatory infiltration in the spinal cord of EAE rats steadily increased from day 10 pi to days 12-14 pi, it was still marked on day 17 pi and it was negligible on day 21 pi. On day 10 pi only mild infiltration was present, and it was mainly localized in the subarachnoid space and around some of the endoneural vessels. At the peak of the disease (days 12-14 pi) the mononuclear infiltration was abundant and it was present also within spinal cord parenchyma.

### LipoRed® Localization

LipoRed® staining was observed in the ovary of both healthy controls and EAE rats at each time point of examination.

No staining was observed at each time point in the spinal cord of healthy rats.

In EAE rats LipoRed® localization within the spinal cord was evidenced already on day 10 pi, even before a massive perivascular inflammatory infiltration was present. The extent of the signal increased until day 14 pi, it was rather stable on day 17 pi and it was clearly evident until day 21 pi (when virtually no more infiltration was present in the spinal cord).

The analysis of serial reconstructions performed at the confocal laser microscope of LipoRed® signal, strongly suggested that LipoRed® was strictly confined within endoneural vessels, where is has frequently the appearance of discrete spots localized at the vessel wall. On day 21 pi rare rhodamine-positive cells were also observed in LipoRed® stained vessels.

### Conclusion

The present study provides evidence for a localization of LipoRed® within the spinal cord of acute EAE Lewis rats and strongly suggests an endoneural localization of the molecule.

The temporal course of LipoRed® staining is related to the course of the EAE. It is, however, noteworthy that the staining already occurs before the pathological observation of inflammatory infiltration.

### 3. Diagnostic and Therapeutic Applications in Human Patients

### 3.1 General Considerations

### 3.2 Treatment of Human Patients

Human treatment protocols using the disclosed formulations is outlined in the following example. Treatment will be of use to prevent and/or treat various human diseases and disorders associated with altered sites in BBB and/or BNB. It is considered to be particularly useful in neurodegenerative diseases, for example, in treating patients with MS.

Prior to application, the formulation can be reconstituted in an aqueous solution in the event that the formulation was freeze dried. The required application volume is calculated from the patient's body weight and the dose schedule.

The formulation may be administered over a short to medium infusion time. The dose level may be determined according to toxicity measurements. Thus, if Grade II toxicity is reached after any single infusion, or at a particular period of time for a steady rate infusion, further doses should be withheld or the steady rate infusion stopped unless toxicity improved. Increasing doses should be administered to groups of patients until approximately 60% of patients show unacceptable Grade III or IV toxicity in any category. Doses that are ⅔ of this value would be defined as the safe dose.

Physical examination and laboratory tests should, of course, be performed before treatment and at intervals of about 3-4 weeks later. Laboratory tests should include complete blood cell counts, serum creatinine, creatine kinase, electrolytes, urea, nitrogen, SGOT, bilirubin, albumin and total serum protein.

Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by the FDA Office of Biologics standards.

The present invention includes a method of delivery of a pharmaceutically effective amount of the inventive formulation of an active agent to a target site such as an altered site of the BBB or BNB of a subject in need thereof. A “subject in need thereof” refers to a mammal, e.g. a human.

The route of administration preferably comprises peritoneal or parenteral, e.g. intravenous administration.

For use with the present invention the “pharmacologically effective amount” of a compound administered to a subject in need thereof will vary depending on a wide range of factors. The amount of the compound will depend upon the size, age, sex, weight, and condition of the patient, as well as the potency of the substance being administered. Having indicated that there is considerable variability in terms of dosing, it is believed that those skilled in the art can, using the present disclosure, readily determine appropriate dosing by first administering extremely small amounts and incrementally increasing the dose until the desired results are obtained. Although the amount of the dose will vary greatly based on factors as described above, in general, the present invention makes it possible to administer substantially smaller amounts of any substance as compared with delivery systems which do not target the altered sites of the BBB and/or BNB.

### 3.3 Comparison of Once- and Twice Weekly EndoTAG®-1 Application Versus Placebo in the Treatment of MS

### Study Design

A controlled, three armed, randomized, open label clinical phase II trial with once or twice weekly administration of lipid complexed paclitaxel (EndoTAG®-1) versus placebo can be performed in patients with relapse-remitting or secondary progressive multiple sclerosis. Progression of the disease is to be monitored by the appearance of new lesions with features of inflammation, which can be detected by gadolinium-enhanced T₁-weighted MRI (Thompson et al., 1992) (McFarland et al., 1992).

### Inclusion Criteria

Eligible patients meet the following criteria:
18-25 years
clinically definite or laboratory supported definite multiple sclerosis (Poser et al., 1983), either relapse-remitting or secondary progressive multiple sclerosis (Lublin and Reingold, 1996)
Kutzke Expanded Disability Status Score between 2 and 6.5
no relapse within the last 30 days
at least three lesions on T₂-weighted magnetic resonance imaging (MRI) of the brain

### Study Procedure and End Points

prior to the start of the treatment patients are randomized into one of the three groups;
prior to treatment unenhanced proton-density, T₂-weighted MRI and gadolinium-enhanced T₁-weighted MRI scans and Kutzke Expanded Disability Status Score are obtained;
prior to administration, dehydrated EndoTAG®-1 in reconstituted in aseptic saline solution suitable for injection, aseptic saline solution is administered as placebo;
EndoTAG®-1 and placebo is administered i.v. with initially 1 ml/min. After 10 min administration speed will be increased to 1.5 ml/min and after further 10 min administration speed will be set to 1.5 ml/min.
   Group I: EndoTAG®-1. 44 mg/m² on day 1 of every week
   Group II: EndoTAG®-1. 44 mg/m² on days 1 and 4 of every week
   Group III: placebo on days 1 and 4 of every week
treatment is pursued for 6 month
MRI scans are performed every 3 month during the treatment and 3 month after the completion of the treatment
Expanded disability Status Score is determined every 3 month during the treatment and 3 month after the completion of the treatment

### Primary Endpoint:

number of new gadolinium-enhanced lesions in T₁-weighted MRI during the time of observation (from the start of the treatment until the last MRI scan 3 month after the completion of the treatment)

### Secondary Endpoint:

changes in the Kutzke Expanded Disability Status Score
time to progression, whereas progression is defined by an objective relapse accompanied by an increase of the EDSS of at least 1

### Further Endpoints:

number of persistent enhancing lesions
volume of enhancing lesions
number of new or enhancing lesions on T₂-weighted MRI

### 3.4 Therapeutic Application of IFN β Loaded to Cationic Colloidal Carriers for MS

The cationic colloidal carriers will be loaded with IFN-β preparations presently used for the treatment of multiple sclerosis. The direct delivery of IFN-β to BBB will allow a significant reduction of IFN-β dosage to ⅓- 1/10 of that commonly used and a concomittant reduction of the formation of IFN-β antibodies.

### 3.5 Radiolabeled Cationic Liposomes for Scintigraphic Detection of Inflammatory Sites on MS

In this study, cationic liposomes are used to determine and localize sites of inflammation of the blood brain barrier within MS. 20 patients having, or suspect of having, MS are selected.

### Groups:

1. Cationic liposomes: DOTAP/DOPC/PEG-DOPE/DTPA-DOPE/linker lipid, (10 mM total lipid concentration)
2. Anionic liposomes (control) DPPG/DOPC/PEG-DOPE/DTPA-DOPE/linker (10 mM total lipid concentration)
3. As a further control gadolinium-enhanced T₁-weighted MRI (Thompson et al., 1992) (McFarland et al., 1992) measurements are performed.

### Liposome Preparation

Liposomes are produced by established standard protocols. Briefly, from the lipid solution in chloroform in a round bottom flask the solvent was evaporated. The resulting lipid film is reconstituted with an aqueous phase comprising 5% (w/w) glucose. The liposomes are extruded 5 times across membranes of 200 nm pores size and sterile filtrated.

Labeling of the liposomes with ⁹⁹Tc is performed by adding the sufficient amount of aqueous Tc solution.

### Protocol:

A maximum amount of 75 mg/m² of total lipid is applied by slow intravenous injection. The dose of ⁹⁹Tc is about 700 MBq.

Imaging is performed 1 hour, 2 hours and 4 hours after application. Scintigraphic and SPECT images from brain and spinal cord regions are taken according to the known standard with apparatus settings adjusted to the probe and the patient. Scintigraphic images are taken in digital format and analyzed by drawing regions of interest in the relevant tissue regions. In addition to the CNS, the activity in blood, lung, liver, kidneys bladder, spleen and muscle is observed.

### 3.6. Comparison of Once and Twice Weekly Endo-MTX Application Versus Placebo in the Treatment of MS

The assessment of Endo-MTX for the treatment of MS can be performed in analogy to Example 3.3. Instead of EndoTAG®-1, Endo-MTX is administered. The administered dose is between 7.5 and 25 mg of methotrexate.

### REFERENCES

Bertolotto, A. (2004). Neutralizing antibodies to interferon beta: implications for the management of multiple sclerosis. Curr Opin Neurol 17, 241-246.
Cao, L., Sun, D., Cruz, T., Moscarello, M. A., Ludwin, S. K., and Whitaker, J. N. (2000). Inhibition of experimental allergic encephalomyelitis in the Lewis rat by paclitaxel. J Neuroimmunol 108, 103-111.
Decher, G. (1997). Fuzzy Nanoassemblies: Toward Layered Polymeric Multicomposites. Science 277, 1232-1237.
Felgner, J. H., Kumar, R., Sridhar, C. N., Wheeler, C. J., Tsai, Y. J., Border, R., Ramsey, P., Martin, M., and Felgner, P. L. (1994). Enhanced gene delivery and mechanism studies with a novel series of cationic lipid formulations. J Biol Chem 269, 2550-2561.
Gold, R., Hartung, H. P., and Toyka, K. V. (2000). Animal models for autoimmune demyelinating disorders of the nervous system. Mol Med Today 6, 88-91.
Hafler, D. A. (2004). Multiple sclerosis. J Clin Invest 113, 788-794.
Kieseier, B. C., and Hartung, H. P. (2003). Current disease-modifying therapies in multiple sclerosis. Semin Neurol 23, 133-146.
Kirk, S., Frank, J. A., and Karlik, S. (2004). Angiogenesis in multiple sclerosis: is it good, bad or an epiphenomenon? J Neurol Sci 217, 125-130.
Krasnici, S., Werner, A., Eichhorn, M. E., Schmitt-Sody, M., Pahernik, S. A., Sauer, B., Schulze, B., Teifel, M., Michaelis, U., Naujoks, K., and Dellian, M. (2003). Effect of the surface charge of liposomes on their uptake by angiogenic tumor vessels. Int J Cancer 105, 561-567.
Lee, M., Cho, Y., Park, J., Chung, H., Jeong, S., Choi, K., Moon, D., Kim, S., Kim, I.-S., and Kwon, I. (2006). Size control of self-assembled nanoparticles by an emulsion/solvent evaporation method. Colloid & Polymer Science 284, 506-512.
Liuzzi, G. M., Mastroianni, C. M., Latronico, T., Mengoni, F., Fasano, A., Lichtner, M., Vullo, V., and Riccio, P. (2004). Anti-HIV drugs decrease the expression of matrix metalloproteinases in astrocytes and microglia. Brain 127, 398-407.
Liuzzi, G. M., Trojano, M., Fanelli, M., Avolio, C., Fasano, A., Livrea, P., and Riccio, P. (2002). Intrathecal synthesis of matrix metalloproteinase-9 in patients with multiple sclerosis: implication for pathogenesis. Mutt Scler 8, 222-228.
Lublin, F. D., and Reingold, S. C. (1996). Defining the clinical course of multiple sclerosis: results of an international survey. National Multiple Sclerosis Society (USA) Advisory Committee on Clinical Trials of New Agents in Multiple Sclerosis. Neurology 46, 907-911.
Mastronardi, F. G., and Moscarello, M. A. (2005). Molecules affecting myelin stability: a novel hypothesis regarding the pathogenesis of multiple sclerosis. J Neurosci Res 80, 301-308.
McDonald, W. I., Compston, A., Edan, G., Goodkin, D., Hartung, H. P., Lublin, F. D., McFarland, H. F., Paty, D. W., Polman, C. H., Reingold, S. C., et al., (2001). Recommended diagnostic criteria for multiple sclerosis: guidelines from the International Panel on the diagnosis of multiple sclerosis. Ann Neurol 50, 121-127.
McFarland, H. F., Frank, J. A., Albert, P. S., Smith, M. E., Martin, R., Harris, J. O., Patronas, N., Maloni, H., and McFarlin, D. E. (1992). Using gadolinium-enhanced magnetic resonance imaging lesions to monitor disease activity in multiple sclerosis. Ann Neurol 32, 758-766.
Poser, C. M., Paty, D. W., Scheinberg, L., McDonald, W. I., Davis, F. A., Ebers, G. C., Johnson, K. P., Sibley, W. A., Silberberg, D. H., and Tourtellotte, W. W. (1983). New diagnostic criteria for multiple sclerosis: guidelines for research protocols. Ann Neurol 13, 227-231.
Schmidt, J., Metselaar, J. M., Wauben, M. H., Toyka, K. V., Storm, G., and Gold, R. (2003). Drug targeting by long-circulating liposomal glucocorticosteroids increases therapeutic efficacy in a model of multiple sclerosis. Brain 126, 1895-1904.
Schnyder, A., and Huwyler, J. (2005). Drug transport to brain with targeted liposomes. NeuroRx 2, 99-107.
Solodin, I., Brown, C. S., Bruno, M. S., Chow, C. Y., Jang, E. H., Debs, R. J., and Heath, T. D. (1995). A novel series of amphiphilic imidazolinium compounds for in vitro and in vivo gene delivery. Biochemistry 34, 13537-13544.
Stuve, O., Chabot, S., Jung, S. S., Williams, G., and Yong, V. W. (1997). Chemokine-enhanced migration of human peripheral blood mononuclear cells is antagonized by interferon beta-1b through an effect on matrix metalloproteinase-9. J Neuroimmunol 80, 38-46.
Thompson, A. J., Miller, D., Youl, B., MacManus, D., Moore, S., Kingsley, D., Kendall, B., Feinstein, A., and McDonald, W. I. (1992). Serial gadolinium-enhanced MRI in relapsing/remitting multiple sclerosis of varying disease duration. Neurology 42, 60-63.
Werring, D. J., Brassat, D., Droogan, A. G., Clark, C. A., Symms, M. R., Barker, G. J., MacManus, D. G., Thompson, A. J., and Miller, D. H. (2000). The pathogenesis of lesions and normal-appearing white matter changes in multiple sclerosis: a serial diffusion MRI study. Brain 123 (Pt 8), 1667-1676.
Yong, V. W. (2005). Metalloproteinases: mediators of pathology and regeneration in the CNS. Nat Rev Neurosci 6, 931-944.
Zahr, A. S., de Villiers, M., and Pishko, M. V. (2005). Encapsulation of drug nanoparticles in self-assembled macromolecular nanoshells. Langmuir 21, 403-410.

1-28. (canceled)

29. A method for diagnosing or treating a neurological inflammatory or degenerative disease comprising administering to a subject a cationic colloidal carrier composition comprising at least one active agent.

30. A method for diagnosing or treating a demyelinating disease, particularly an inflammatory demyelinating disease, comprising administering to a subject a cationic colloidal carrier composition comprising at least one active agent.

31. The method of claim 29, wherein the disease is selected from diseases in the central nervous system (CNS) and diseases in the peripheral nervous system (PNS).

32. The method of claim 29, wherein the disease is multiple sclerosis.

33. The method of claim 29, wherein the disease is Guillain-Barré syndrome.

34. The method of claim 29, wherein the disease is experimental autoimmune encephalomyelitis or experimental autoimmune neuritis.

35. A method for targeting delivery of an active agent to inflammatory or altered sites of the blood-brain barrier (BBB) and/or blood-nerve barrier (BNB) vasculature comprising administering to a subject a cationic colloidal carrier composition comprising the active agent.

36. The method of claim 29, wherein the active agent is a therapeutic agent.

37. The method of claim 29, wherein the active agent is an inhibitor of angiogenesis or an activator of angiogenesis.

38. The method of claim 37, wherein the inhibitor of angiogenesis is a taxane, preferably paclitaxel or docetaxel.

39. The method of claim 29, wherein the composition comprises paclitaxel in an amount of at least about 2 mole % to about 8 mole %, preferably from at least 2.5 mole % to about 3.5 mole % of total carrier components.

40. The method of claim 29, wherein the active agent is an immunomodulatory cytostatic agent, a cytokine or cytokine inhibitor, an immunosuppressive antibody, a corticosteroide or a combination thereof.

41. The method of claim 29, wherein the active agent is interferon-β or a derivative or active fragment thereof, azathioprine, cyclophosphamide, mitoxantrone, methotrexate, an anti-α4 integrin antibody, glatiramer acetate, prednisolone or a combination thereof.

42. The method of claim 29, wherein the active agent is a diagnostic agent.

43. The method of claim 42, wherein the diagnostic agent is a metal ion or a metal ion chelate, preferably gadolinium chelate.

44. The method of claim 29, wherein the active agent is a combination of a therapeutic agent and a diagnostic agent.

45. The method of claim 29, wherein the cationic carrier composition comprises a colloidal carrier particle in the size range between about 1 and about 5000 nm, more preferably between about 10 and about 1000 nm.

46. The method of claim 29, wherein the cationic colloidal carrier composition comprises a liposomal preparation.

47. The method of claim 29, wherein the cationic colloidal preparation comprises at least one cationic lipid from of at least about 30 mol %, more preferably at least about 50 mol % and optionally at least one neutral and/or anionic lipid in an amount of up to about 70 mole %, preferably up to about 55 mole % of total carrier components and an active agent.

48. The method of claim 29, wherein the cationic colloidal carrier preparation comprises DOTAP, DOPC and paclitaxel, preferably in a molar ratio of 50:47:3.

49. The method of claim 29, wherein the cationic colloidal composition has a zeta potential in the range of about +20 mV to 100 mV, preferably at least about +30 mV in about 0.05 mM KCl solution at about pH 7.5.

50. The method of claim 29, wherein the liposomal preparation comprises liposomes having an average particle diameter from about 25 nm to about 500 nm, preferably about 100 nm to about 300 nm.

51. The method of claim 29, wherein the cationic colloidal composition is for systemic, preferably intravenous administration.

52. The method of claim 29, wherein the cationic carrier composition for administration to a mammal, particularly to a human patient.

53. The method of claim 29, wherein the cationic carrier composition is for administration in combination with at least one further active agent.

54. The method of claim 53, wherein the further active agent is a therapeutic agent.

55. The method of claim 29, wherein the cationic colloidal composition is for administration as a pharmaceutical composition, which additionally comprises a physiologically acceptable carrier.
